(19) United States
(12) Patent Application Publication
(10) Pub. No.: US 2019/0183827 A1
(43) Pub. Date: Jun. 20, 2019

(54) COMPOSITION FOR IMPROVING MUSCULAR ENDURANCE

(71) Applicant: AJINOMOTO CO., INC., Tokyo (JP)

(72) Inventors: Kyoko MIURA, Kawasaki-shi (JP); Katsuya SUZUKI, Kawasaki-shi (JP)

(73) Assignee: AJINOMOTO CO., INC., Tokyo (JP)

(21) Appl. No.: 16/283,064

(22) Filed: Feb. 22, 2019

Related U.S. Application Data
(63) Continuation of application No. PCT/JP2017/031536, filed on Sep. 1, 2017.

(30) Foreign Application Priority Data
Sep. 2, 2016 (JP) 2016-171989

(51) Int. Cl. A61K 31/198; A23L 33/175; A61K 31/4172; A61K 31/405; A61P 21/00

(52) U.S. Cl. CPC A61K 31/198; A23L 33/175; A23V 2002/00; A61K 31/405; A61P 21/00; A61K 31/4172

(57) ABSTRACT

Compositions which contain one or more kinds of essential amino acids other than leucine and not less than 35 mol % of leucine, relative to the total content of essential amino acids, are useful for improving muscular endurance, can be ingested safely, and can efficiently improve muscular endurance conveniently in a short period of time.

### CROSS REFERENCES TO RELATED APPLICATIONS

This application is a continuation of International Patent Application No. PCT/JP2017/031536, filed on Sep. 1, 2017, and claims priority to Japanese Patent Application No. 2016-171989, filed on Sep. 2, 2016, both of which are incorporated herein by reference in their entireties.

### BACKGROUND OF THE INVENTION

### Field of the Invention

The present invention relates to compositions for improving muscular endurance and to methods of improving muscular endurance.

### Discussion of the Background

In daily life, to actually move the body and to sustain the action, it is important that the muscles can continuously demonstrate their strength, that is, maintain and improve the muscle endurance. For this end, it is effective to carry out training to maintain and improve muscle endurance and to provide particular nutritional supplementation to maintain and improve muscle endurance before exercise.

Improvement of muscle endurance in exercise is necessary for athletes to improve competitive performance. Also, even for non-athletes, maintaining and improving muscle endurance are important in exercises to maintain health and live an active life. Particularly, for patients having diseases such as lifestyle-related diseases and the like and the elderly people, continuation of exercise is often important for treatment of the diseases and prevention of aging.

To improve muscular endurance which is important for continuation of exercise, it is usually necessary to repeat long training since training does not provide a sufficient effect in a short period of time. However, continuation of training is often difficult mentally and physically for patients with disease and elderly people with declining physical strength.

Therefore, to efficiently improve muscle endurance in exercise, advanced nutritional supplementation is a beneficial method and is very important not only for top-level athletes but also for patients and the like who are receiving exercise therapy.

From the aspect of the prevention of doping, generally, the muscular endurance of athletes is desirably improved by ingestion of nutrients used as food. In addition, patients under exercise therapy and elderly people are highly likely taking some kind of medicine already. Thus, it is desirable for them to take nutrients to improve muscle endurance.

Glycogen loading which accumulates glycogen in muscle is known as a method for improving muscle endurance by nutrient ingestion.

Glycogen loading aims to increase glycogen in muscle more effectively by ingesting low carbohydrate diet in advance, doing intense exercise to drastically decrease the amount of glycogen in the muscle, and switching to a high carbohydrate diet.

However, this method needs to be performed for a long term and requires complicated processes for preparation of meals with calculated nutrition value, setting of exercise program to deplete glycogen, and the like.

Therefore, a method based on the ingestion of nutrients, that can improve muscle endurance easily and in a short time is desired.

On the other hand, it is known that ingestion of branched chain amino acids such as leucine, isoleucine and valine, among amino acids as nutrients constituting muscle proteins, after exercise reduces muscle damage and muscle fatigue due to excessive exercise.

WO 2013/021891, which is incorporated herein by reference in its entirety, describes that a composition containing a high content (35 mol % to 66 mol %) of leucine and a given amount of essential amino acid other than leucine has an action to promote recovery from muscle fatigue.

However, the effect of the ingestion of branched chain amino acid and essential amino acid before and during exercise on muscular endurance is not known yet.

Calders et al. reported that intraperitoneal administration of branched chain amino acid before exercise prolonged duration of running exercise in animal experiments (see Calders et al.; Medicine & Science in Sports & Exercise (1999), 31(4), 583-587, which is incorporated herein by reference in its entirety). In addition, Chen-Kang et al. reported that ingestion of branched chain amino acid and arginine decreased sprint time in running exercise after a certain exercise in the study of human (see Chen-Kang et al.; PLOS One (2015), 10(3), e0121866/1-e0121866/13, which is incorporated herein by reference in its entirety).

However, since the exercises considered in the above-mentioned documents are running exercises using the whole body and the sprint time is an exercise performed in an extremely short time, the results reported in the above-mentioned documents do not reflect the muscular endurance. That is, it is not possible to assume, from the results of the duration of the running time and sprint time, the influence of branched chain amino acid and essential amino acid on the improvement of muscle endurance in exercises using specific muscles such as resistance movement and the like.

Recovery of muscle fatigue and improvement of muscle endurance are physiologically completely different reactions and it is not easy to assume whether branched chain amino acid and essential amino acid can improve muscle endurance from the disclosure of WO 2013/021891.

### SUMMARY OF THE INVENTION

Accordingly, it is one object of the present invention to provide novel compositions for improving muscular endurance that are constituted of nutrients, can be ingested or administered safely and continuously, and can efficiently improve muscular endurance conveniently in a short period of time.

It is another object of the present invention to provide novel methods of improving muscular endurance by administering such a composition.

These and other objects, which will become apparent during the following detailed description, have been achieved by the inventors' discovery that a composition containing leucine at a high content and one or more kinds of essential amino acids other than leucine improves muscle endurance by suppressing a decrease in muscle force caused by exercise load on the muscle, and further that the composition affords a superior muscular endurance-improving effect by simply ingesting or administering one time before exercise.

That is, the present invention provides the following.

(1) A composition for improving muscular endurance comprising one or more kinds of essential amino acids other than leucine and not less than 35 mol % of leucine relative to the total content of essential amino acids.

(2) The composition of (1), wherein the content of leucine is 35 mol % to 66 mol % relative to the total content of essential amino acids.

(3) The composition of (1) or (2), wherein one or more kinds of essential amino acids other than leucine are isoleucine and valine.

(4) The composition of (3), wherein a molar composition ratio of the content of each amino acid relative to the total content of essential amino acids falls within the following numerical values:

leucine 35 mol % to 66 mol %

isoleucine 5 mol % to 15 mol %

valine 5 mol % to 15 mol %.

(5) The composition of (1) or (2), wherein one or more kinds of essential amino acids other than leucine are isoleucine, valine, threonine, lysine, and phenylalanine.

(6) The composition of (5), wherein a molar composition ratio of the content of each amino acid relative to the total content of essential amino acids falls within the following numerical values:

leucine 35 mol % to 66 mol %

isoleucine 5 mol % to 15 mol %

valine 5 mol % to 15 mol %

threonine 7 mol % to 14 mol %

lysine 8 mol % to 16 mol %

phenylalanine 2.5 mol % to 8 mol %.

(7) The composition of (1) or (2), wherein one or more kinds of essential amino acids other than leucine are isoleucine, valine, threonine, lysine, methionine, histidine, phenylalanine, and tryptophan.

(8) The composition of (7), wherein a molar composition ratio of the content of each amino acid relative to the total content of essential amino acids falls within the following numerical values:

leucine 35 mol % to 66 mol %

isoleucine 5 mol % to 15 mol %

valine 5 mol % to 15 mol %

threonine 7 mol % to 14 mol %

lysine 8 mol % to 16 mol %

methionine 2 mol % to 10 mol %

histidine 0.1 mol % to 3.5 mol %

phenylalanine 2.5 mol % to 8 mol %

tryptophan 0.1 mol % to 2 mol %.

(9) The composition of any of (1) to (8), wherein the composition is used for improving muscular endurance against resistance exercise load.

(10) The composition of (9), wherein the resistance exercise is concentric exercise.

(11) The composition of any of (1) to (10), wherein the composition is ingested or administered once before exercise.

(12) The composition of any of (1) to (11), wherein the composition is a pharmaceutical product.

(13) The composition of any of (1) to (11), wherein the composition is a food.

(14) A method for improving muscular endurance, comprising ingestion by or administration to a subject animal in need of improvement in muscular endurance of an effective amount of a composition comprising one or more kinds of essential amino acids other than leucine and not less than 35 mol % of leucine relative to the total content of essential amino acids.

(15) The method of (14), wherein a content of leucine in the composition is 35 mol % to 66 mol % relative to the total content of essential amino acids.

(16) The method of (14) or (15), wherein one or more kinds of essential amino acids other than leucine are isoleucine and valine.

(17) The method of (14) or (15), wherein one or more kinds of essential amino acids other than leucine are isoleucine, valine, threonine, lysine, and phenylalanine.

(18) The method of (14) or (15), wherein one or more kinds of essential amino acids other than leucine are isoleucine, valine, threonine, lysine, methionine, histidine, phenylalanine, and tryptophan.

(19) The method of any of (14) to (18), wherein the method is used for improving muscular endurance against resistance exercise load.

(20) The method of (19), wherein the resistance exercise is concentric exercise.

(21) The method of any of (14) to (20), wherein the method comprises ingestion or administration once before exercise.

### Effect of the Invention

According to the present invention, a composition for improving muscular endurance that can efficiently improve muscular endurance conveniently in a short period of time can be provided.

That is, the composition for improving muscular endurance of the present invention can improve muscle endurance by suppressing a decrease in muscle force caused by exercise load.

Furthermore, the composition for improving muscular endurance of the present invention affords a good effect of suppressing a decrease in muscle force and a muscular endurance improving effect against exercise load by ingestion or administration once before exercise.

The composition for improving muscular endurance of the present invention is particularly effective for suppressing a decrease in muscle force and improving muscular endurance when a resistance exercise is performed in which the load is concentrated on the target muscle, and further effective for suppressing a decrease in muscle force and improving muscular endurance when concentric exercise is performed.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the effect of the composition for improving muscular endurance of Example 1 of the present invention on muscle endurance on loading an eccentric exercise load. In the Figure, group AA shows the group orally administered with the composition of Example 1.

FIG. 2 shows the effect of the composition for improving muscular endurance of Example 1 of the present invention on muscle endurance on loading a concentric exercise load. In the Figure, group AA shows the group orally administered with the composition of Example 1. In addition, “*” shows significance at P<0.05 and “**” shows significance at P<0.01.

FIG. 3 shows the effect of the composition for improving muscular endurance of Example 1 of the present invention on muscle endurance on loading an eccentric exercise load under different exercise load conditions. In the Figure, group AA shows the group orally administered with the composition of Example 1.

FIG. 4 shows the effect of the composition for improving muscular endurance of Example 2 of the present invention on muscle endurance on loading a concentric exercise load. In the Figure, group AB shows the group orally administered with the composition of Example 2.

### DETAILED DESCRIPTION OF THE PREFERRED EMBODIMENTS

The composition for improving muscular endurance of the present invention (hereinafter to be also referred to as “the composition of the present invention” in the present specification) contains one or more kinds of essential amino acids other than leucine (i.e., one or more kinds selected from the group consisting of isoleucine, valine, threonine, lysine, methionine, histidine, phenylalanine and tryptophan), and not less than 35 mol % of leucine relative to the total content of essential amino acids.

The “leucine” and “essential amino acid other than leucine” used may be any of an L form, a D form and a DL form. An L form and a DL form are preferably used, and an L form is more preferably used.

The “leucine” and “essential amino acid other than leucine” can be used not only in a free form but also a salt form. The term “leucine” and “essential amino acid other than leucine” in the present specification are concepts each encompassing even a salt. The salt form is not particularly limited as long as it is a pharmacologically acceptable salt, and acid addition salt, salt with base and the like can be mentioned.

Concrete examples include salts with inorganic bases, salts with organic bases, salts with inorganic acids, salts with organic acids, salts with amino acid and the like.

Examples of the salts with inorganic bases include salts with alkali metals such as lithium, sodium, potassium and the like, salts with alkaline earth metals such as magnesium, calcium and the like, ammonium salt and the like.

Examples of the salts with organic bases include salts with alkanolamine such as monoethanolamine, diethanolamine, triethanolamine and the like, salts with heterocyclic amine such as morpholine, piperidine and the like, and the like.

Examples of the salts with inorganic acids include salts with hydrohalic acid (hydrochloric acid, hydrobromic acid, hydroiodic acid etc.), sulfuric acid, nitric acid, phosphoric acid and the like.

Examples of the salts with organic acids include salts with monocarboxylic acid such as formic acid, acetic acid, propanoic acid and the like; salts with saturated dicarboxylic acid such as oxalic acid, malonic acid, malic acid, succinic acid and the like; salts with unsaturated dicarboxylic acid such as maleic acid, fumaric acid and the like; salts with tricarboxylic acid such as citric acid and the like; salts with keto acid such as α-ketoglutaric acid and the like.

Examples of the salts with amino acid include salts with aliphatic amino acid such as glycine, alanine and the like; salts with aromatic amino acid such as tyrosine and the like; salts with basic amino acid such as arginine and the like; salts with acidic amino acid such as aspartic acid, glutamic acid and the like; salts with amino acid forming lactam such as pyroglutamic acid and the like; and the like.

The above-mentioned salts may each be a hydrate (hydrated salt), and examples of the hydrate include 1 hydrate to 6 hydrate and the like.

In the present invention, one kind each of “leucine” and “essential amino acid other than leucine” in the above-mentioned free form or a salt form may be used singly, or two or more kinds thereof may be used in combination.

For the object of the present invention, a free form, hydrochloride or the like of each of “leucine” and “essential amino acid other than leucine” is preferable.

In the present invention, the above-mentioned each amino acid in a free form or in the form of a salt to be used may be extracted from animals, plants or the like, which are naturally present, and purified, or obtained by a chemical synthesis method, a fermentation method, an enzyme method or a gene recombinant method. Commercially available products provided by each company may also be utilized.

In the composition of the present invention, leucine is contained at a high content of not less than 35 mol % relative to the total content of essential amino acids.

In the present specification, the content of each amino acid such as leucine in the composition of the present invention when it is contained in the form of a salt is shown by the content converted to that of a free form.

From the aspect of a muscular endurance improving effect, the content of leucine is preferably 35 mol % to 66 mol %, more preferably 35 mol % to 57 mol %, further preferably 35 mol % to 50 mol %, relative to the total content of essential amino acids.

The content of isoleucine contained as essential amino acid other than leucine is preferably 5 mol % to 15 mol % relative to the total content of the essential amino acids.

The content of valine contained as essential amino acid other than leucine is preferably 5 mol % to 15 mol % relative to the total content of the essential amino acids.

The content of threonine contained as essential amino acid other than leucine is preferably 7 mol % to 14 mol % relative to the total content of the essential amino acids.

The content of lysin contained as essential amino acid other than leucine is preferably 8 mol % to 16 mol % relative to the total content of the essential amino acids.

The content of methionine contained as essential amino acid other than leucine is preferably 2 mol % to 10 mol % relative to the total content of the essential amino acids.

The content of histidine contained as essential amino acid other than leucine is preferably 0.1 mol % to 3.5 mol % relative to the total content of the essential amino acids.

The content of phenylalanine contained as essential amino acid other than leucine is preferably 2.5 mol % to 8 mol % relative to the total content of the essential amino acids.

The content of tryptophan contained as essential amino acid other than leucine is preferably 0.1 mol % to 2 mol % relative to the total content of the essential amino acids.

From the aspect of a muscular endurance improving effect, the composition of the present invention preferably contains isoleucine and valine at the above-mentioned contents as essential amino acids other than leucine, more preferably contains isoleucine, valine, threonine, lysine, and phenylalanine at the above-mentioned contents as essential amino acids other than leucine, and further preferably contains isoleucine, valine, threonine, lysine, methionine, histidine, phenylalanine, and tryptophan at the above-mentioned contents as essential amino acids other than leucine.

The composition of the present invention optionally contains other nutrition components besides the above-mentioned essential amino acids.

Examples of other nutrition component include non-essential amino acids such as serine, glutamine, arginine, cystine and the like; carbohydrates such as glucose, dextran, starch and the like; lipids such as purified soybean oil, purified egg-yolk lecithin and the like; proteins such as casein, whey protein and the like; vitamins such as vitamin A (retinol, retinal, retinoic acid etc.), vitamin B group (vitamin B₁ (thiamine), vitamin B₂ (riboflavin), niacin (nicotinic acid, nicotinamide), vitamin B₆ (pyridoxal, pyridoxamine, pyridoxine), biotin, folic acid, vitamin B₁₂ (cyanocobalamin, hydroxocobalamin) etc.), vitamin C (ascorbic acid etc.), vitamin D (cholecalciferol, ergocalciferol etc.), vitamin E (tocopherol, tocotrienol etc.), vitamin K (phylloquinone, menaquinone, menadione etc.) and the like; minerals such as sodium chloride, potassium chloride, calcium chloride, phosphoric acid dipotassium, magnesium sulfate and the like, and the like, with preference given to non-essential amino acids.

One kind of the above-mentioned nutrition component may be used singly, or two or more kinds thereof may be used in combination.

The composition of the present invention can be formulated into various forms such as liquids such as solution, suspension, emulsion and the like; semi-solid such as gel, cream and the like; solid such as powder, granule, tablet, capsule and the like, and the like by adding other nutrition components and pharmaceutically acceptable additives to leucine and essential amino acid other than leucine as necessary and according to a formulating means well known in the field of preparations, for example, the methods described in the Japanese Pharmacopoeia XVII General Rules for preparations [3] Monographs for Preparations, which is incorporated herein by reference in its entirety, and the like.

The above-mentioned pharmaceutically acceptable additive can be appropriately selected according to the form of the composition of the present invention and, for example, excipient, binder, disintegrant, lubricant, coating agent, base, solvent, solubilizing agents, solubilizer, emulsifier, dispersing agent, suspending agent, stabilizer, thickener, soothing agent, isotonicity agent, pH adjuster, antioxidant, antiseptic, preservative, corrigent, sweetening agent, flavor, colorant and the like can be mentioned.

To be specific, examples of the excipient include magnesium carbonate, saccharides (glucose, lactose, cornstarch etc.), sugar alcohol (sorbitol, mannitol etc.) and the like.

Examples of the binder include gelatin, pregelatinized starch, partly pregelatinized starch, cellulose and a derivative thereof (crystalline cellulose, hydroxypropylcellulose, etc.) and the like.

Examples of the disintegrant include crospovidone, povidone, crystalline cellulose and the like.

Examples of the lubricant include talc, magnesium stearate and the like.

Examples of the coating agent include methacrylic acid. methyl methacrylate copolymer, methacrylic acid.ethyl acrylate copolymer, methyl methacrylate.butyl methacrylate.methacrylic acid dimethylaminoethyl copolymer, ethyl acrylate.methyl methacrylate.methacrylic acid trimethylammonium chloride ethyl copolymer and the like.

Examples of the base include animal and plant fats and oils (olive oil, cacao butter, beef tallow, sesame oil, hydrogenated oil, castor oil etc.), wax (Carnauba wax, beeswax etc.), polyethylene glycol and the like.

Examples of the solvent include purified water, water for injection, monovalent alcohol (ethanol etc.), polyhydric alcohol (glycerol etc.) and the like.

Examples of the solubilizing agent include propylene glycol, medium-chain triglyceride and the like.

Examples of the solubilizer, emulsifier, dispersing agent and suspending agent include surfactant and the like such as sorbitan fatty acid ester, glycerol fatty acid ester, polyoxyethylene sorbitan fatty acid ester (polysorbate 20 etc.), polyoxyethylene hydrogenated castor oil, sucrose fatty acid ester and the like.

Examples of the stabilizer include adipic acid, β-cyclodextrin, ethylenediamine, sodium edetate and the like.

Examples of the thickener include water-soluble polymer (sodium polyacrylate, carboxyvinyl polymer etc.), polysaccharides (sodium alginate, xanthan gum, tragacanth etc.) and the like.

Examples of the soothing agent include ethyl aminobenzoate, chlorobutanol, propylene glycol, benzyl alcohol and the like.

Examples of the isotonicity agent include potassium chloride, sodium chloride, sorbitol, saline and the like.

Examples of the pH adjuster include hydrochloric acid, sulfuric acid, acetic acid, citric acid, lactic acid, sodium hydroxide, potassium hydroxide and the like.

Examples of the antioxidant include dibutylhydroxytoluene (BHT), butylhydroxyanisole (BHA), dl-α-tocopherol, erythorbic acid and the like.

Examples of the antiseptic and preservative include paraben (methylparaben etc.), benzyl alcohol, sodium dehydroacetate, sorbic acid and the like.

Examples of the corrigent include ascorbic acid, erythritol, L-sodium glutamate and the like.

Examples of the sweetening agent include aspartame, licorice extract, saccharin and the like.

Examples of the flavor include 1-menthol, d-camphor, vanillin and the like.

Examples of the colorant include tar pigment (Food Color Red No. 2, Food Color Blue No. 1, Food Color yellow No. 4, etc.), inorganic pigment (red ferric oxide, yellow iron oxide, black iron oxide, etc.), natural dye (turmeric extract, β-carotene, sodium copper-chlorophyllin, etc.) and the like.

In the present invention, one or more kinds of the above-mentioned additive can be used.

The daily ingestion amount or dose of the composition of the present invention is appropriately determined according to the condition or symptom, sex, age of the target to be applied to (hereinafter to be also referred to as the “application target” in the present specification), the form of the composition of the present invention, administration method and the like. When the application target is a human adult, it is generally 10 mg/kg body weight/day to 1000 mg/kg body weight/day, preferably 20 mg/kg body weight/day to 700 mg/kg body weight/day, more preferably 30 mg/kg body weight/day to 400 mg/kg body weight/day, as the total amount of leucine and essential amino acid other than leucine (the total amount converted to the amount of free form).

The above-mentioned amount can be ingested or administered at once or in several portions (e.g., 2 to 3 portions) per day.

From the aspect of muscular endurance-improving effect, the composition of the present invention in the above-mentioned amount is preferably ingested or administered once before exercise. Being “ingested or administered before exercise” generally refers to ingestion or administration immediately before to 360 min before start of exercise, preferably ingestion or administration immediately before to 120 min before start of exercise.

When the frequency of exercise is high such as when exercise is performed every day, and the like, or when exercise is continued for a certain period of time, ingestion or administration of the composition of the present invention before exercise can be continuously performed every time the exercise is carried out.

The composition of the present invention can be formulated as a unit package form. In the present specification, the “unit package form” means a form of one or more units with a particular amount (e.g., ingestion amount or dose per one time etc.) as one unit is/are packed in one container or package. For example, a unit package form with ingestion amount or dose per one time as one unit is referred to as “unit package form for ingestion amount or dose per one time”. A container or package used for the unit package form can be appropriately selected according to the form and the like of the composition of the present invention. For example, paper container or bag, plastic container or bag, pouch, aluminum can, steel can, glass bottle, pet bottle, PTP (press through pack) package sheet and the like can be mentioned.

The application target of the composition of the present invention includes, for example, mammals (e.g., human, monkey, mouse, rat, guinea pig, hamster, rabbit, cat, dog, bovine, horse, donkey, swine, sheep, etc.), birds (e.g., duck, chicken, goose, turkey, etc.) and the like.

When the composition of the present invention is applied to an application subject animal (hereinafter to be also simply referred to as “subject animal”) other than human, the ingestion amount or dose of the composition of the present invention can be appropriately set according to the kind, sex, body weight and the like of the subject animal.

The composition of the present invention can improve muscular endurance by suppressing a decrease in muscle force against exercise load. As used herein, the “muscle force” means a force that muscles exert in one contraction and the “muscular endurance” means ability of muscles to repeat contraction.

The composition of the present invention can improve, as mentioned above, muscular endurance well by ingestion or administration thereof once before exercise, and muscular endurance can be improved conveniently in a short period without requiring long-term practice or complex process like glycogen loading.

In addition, the composition of the present invention is effective for suppressing a decrease in muscle force and improving muscle endurance when resistance exercise is performed, and particularly effective for suppressing a decrease in muscle force and improving muscle endurance when concentric exercise is performed.

As used herein, “resistance exercise” is an exercise for repeatedly performing an action to place resistance on the target muscle, such as squat, push-ups, dumbbell exercise, and the like and includes exercises seen in daily actions such as ascending and descending stairs.

The “concentric exercise” is an exercise causing concentric contraction of muscle among the resistance movements. Among the resistance exercises, an exercise causing eccentric contraction of muscles is referred to as “eccentric exercise”.

Therefore, the composition of the present invention is preferably ingested by or administered to not only athletes requiring improvement of muscular endurance but also patients under exercise therapy or rehabilitation who are in need of continued exercise, elderly people and middle-aged people who are required to maintain muscle force and muscular endurance, and suppress decrease therein.

The composition of the present invention can be provided as a pharmaceutical product (hereinafter to be also referred to as “the pharmaceutical product of the present invention” in the present specification) directly or by further adding the above-mentioned pharmaceutically acceptable additives.

The pharmaceutical product of the present invention can have a dosage form of oral preparation such as tablet, coating tablet, chewable tablet, pill, (micro)capsule, granule, fine granule, powder, elixir, lemonade, syrup, suspension, emulsion, oral jelly and the like, injection such as solution, suspension, emulsion and the like, solid injection to be used by dissolving or suspending when in use, injectable preparation such as transfusion, sustainable injection and the like, tubal liquid, and the like.

The pharmaceutical product of the present invention is preferably administered to athletes requiring improvement of muscular endurance, patients requiring continuation of exercise therapy and rehabilitation, and elderly people and middle-aged people showing a decrease in muscle force and muscular endurance.

The pharmaceutical product of the present invention is administered to the above-mentioned application target such that the total amount per day of leucine and essential amino acid other than leucine is the above-mentioned dose per day.

In addition, from the aspect of the muscular endurance improving effect, the pharmaceutical product of the present invention is preferably administered once before exercise.

Furthermore, the composition of the present invention can be ingested by adding to various foods. The food to which the composition of the present invention is added is not particularly limited, and may be any as long as it is a food in the form generally served for meals or dessert.

For example, the composition of the present invention is added to drinks such as beverage water and the like, and a suitable flavor is added when desired, whereby a drink can be provided.

More specifically, the composition of the present invention can be added, for example, to beverage water such as fruit juice drinks, sport drinks and the like; dairy products such as milk, yogurt and the like; confectionery such as jelly, chocolate, candy and the like, and the like.

The composition of the present invention is preferably added to the above-mentioned various foods in amounts to be ingested per day such that the total amount of leucine and essential amino acid other than leucine is the above-mentioned dose per day.

While the food to which the composition of the present invention is added can be ingested as usual meal or dessert, it is preferably ingested once before exercise from the aspect of muscular endurance-improving effect.

The composition of the present invention can be provided as a food (hereinafter to be also referred to as “the food of the present invention” in the present specification) directly or by adding general food additives as necessary and according to a general food production technique.

The food of the present invention can be prepared as various forms such as liquid, suspension, emulsified liquid, gel, cream, powder, granule, sheet, capsule, tablet and the like.

Furthermore, the food of the present invention can be prepared as various food forms such as beverage water (fruit juice drinks, sport drinks, coffee drinks, tea drinks, etc.), dairy product (lactic fermenting beverage, fermented milk, butter, cheese, yogurt, processed milk, defatted milk, etc.), meat product (ham, sausage, hamburger, etc.), fish meat processed seafood paste product (fish cake, tube-shaped fish sausage, deep-fried ball of fish paste, etc.), egg product (rolled Japanese-style omelette, steamed egg custard, etc.), confectionery (cookie, jelly, chewing gum, candy, snack food, frozen dessert, etc.), bread, noodles, pickle, dried fish, food boiled in soy sauce, soup, seasoning and the like by adding the composition of the present invention to various food starting materials and adding general food additives as necessary. It may also be a bottled food, canned food or retort pouch food.

As the above-mentioned food additive, manufacturing agent (brine, binding agent, etc.), thickening stabilizer (xanthan gum, sodium carboxymethylcellulose, etc.), gelling agent (gelatin, agar, carrageenan, etc.), gum base (vinyl acetate resin, jelutong, chicle, etc.), emulsifier (glycerol fatty acid ester, sucrose fatty acid ester, saponin, lecithin, etc.), preservative (benzoic acid, sodium benzoate, sorbic acid, potassium sorbate, ε-polylysine, etc.), antioxidant (ascorbic acid, erythorbic acid, catechin, etc.), glazing agent (shellac, paraffin wax, beeswax, etc.), fungicide (thiabendazole, fludioxonil, etc.), leavening agent (sodium hydrogen carbonate, glucono δ-lactone, alum, etc.), sweetener (aspartame, acesulfame potassium, licorice extract, etc.), bittering agent (caffeine, naringin, worm wood extract, etc.), acidulant (citric acid, tartaric acid, lactic acid, etc.), seasoning (L-sodium glutamate, disodium 5′-inosinate, etc.), colorant (annatto dye, turmeric dye, gardenia dye, etc.), flavor (synthetic flavor such as ethyl acetoacetate, anisealdehyde and the like, natural flavor such as orange, lavender and the like) and the like can be mentioned.

In the present invention, one or more kinds of the above-mentioned food additives can be used.

The food of the present invention can be preferably ingested by athletes who are required to improve muscular endurance, patients requiring exercise therapy and continuous rehabilitation, elderly people and middle-aged people showing a decrease in muscle force and muscular endurance and the like.

The food of the present invention can be preferably ingested widely by those who desire to maintain muscle force and improve muscular endurance such as those who are not athletes but routinely exercise well, middle-aged peoples who desire to maintain muscle force and muscular endurance or desire to prevent lowering thereof, and the like.

Therefore, the food of the present invention can also be provided as food with health claims such as food for specified health uses, food with nutrient function claims, indicated functional food and the like, special purpose foods such as food for sick people, food for the elderly and the like, health supplement and the like for the maintenance or improvement of muscle force and muscular endurance.

The food of the present invention is preferably ingested by the above-mentioned application target such that the total amount of leucine and essential amino acid other than leucine per day is the above-mentioned ingestion amount per day.

In addition, while the food of the present invention can be ingested as usual meal or dessert, it is preferably ingested once before exercise from the aspect of muscular endurance-improving effect.

Furthermore, the present invention also provides a method for improving muscular endurance of a subject animal in need of improvement of the muscular endurance (hereinafter to be also referred to as “the method of the present invention” in the present specification).

The method of the present invention comprises ingestion or administration of a composition containing not less than 35 mol % of leucine relative to the total content of essential amino acids and one or more kinds of essential amino acids other than leucine to a subject animal in need of improvement of muscular endurance in an amount effective for improving muscular endurance of the subject animal.

As one or more kinds of essential amino acids other than leucine to be contained in the composition to be ingested by or administered to the subject animal, isoleucine and valine are preferable, isoleucine, valine, threonine, lysine and phenylalanine are more preferable, and isoleucine, valine, threonine, lysine, methionine, histidine, phenylalanine, and tryptophan are further preferable.

The leucine and essential amino acid other than leucine, as well as the contents thereof in the composition to be ingested by or administered to the subject animal are as mentioned above.

The subject animal in the method of the present invention includes mammal (e.g., human, monkey, mouse, rat, guinea pig, hamster, rabbit, cat, dog, bovine, horse, donkey, swine, sheep, etc.), birds (e.g., duck, chicken, goose, turkey, etc.) and the like.

The method of the present invention particularly suppresses a decrease in muscle force when performing resistance exercise that concentrates the load on target muscle, and thus effectively improves muscular endurance. Furthermore, the method is effective for suppressing a decrease in muscle force and improving muscular endurance when performing concentric exercise.

In the case of human, the method of the present invention can be widely applied to those who wish to maintain muscle force and improve muscular endurance, it is particularly preferably applied to athletes who are required to improve muscular endurance, patients requiring exercise therapy and continuous rehabilitation, elderly people and middle-aged people showing a decrease in muscle force and muscular endurance and the like.

While the effective amount of leucine and essential amino acid other than leucine in the method of the present invention is determined according to the kind, age, symptom, condition and the like of the subject animal, an amount similar to the above-mentioned ingestion amount or dose of the composition of the present invention for a human or a subject animal other than human can be ingested or administered at the frequency mentioned above.

The above-mentioned effective amounts of leucine and essential amino acid other than leucine are preferably ingested by or administered to the subject animal once before exercise.

When the frequency of exercise is high such as when the subject animal performs exercise every day, and the like, or when exercise is continued for a certain period of time, the above-mentioned ingestion or administration before exercise can be continuously performed every time the exercise is carried out.

The ingestion or administration method of leucine and essential amino acid other than leucine in the method of the present invention includes oral administration, enteral tube administration, administration by infusion and the like. Oral administration is preferable since convenient ingestion is possible without the need to perform under the guidance and supervision of a doctor at a medical institution.

Other features of the invention will become apparent in the course of the following descriptions of exemplary embodiments which are given for illustration of the invention and are not intended to be limiting thereof.

### EXAMPLES

### Example 1. Composition for Improving Muscular Endurance

A given amount of each component was weighed to afford the composition shown in Table 1 and mixed to prepare the composition for improving muscular endurance of Example 1 (hereinafter to be referred to as “the composition of Example 1”).

TABLE 1

| | molar composition ratio (mol %) |
|---|---|
| | relative to total content of |
| component | essential amino acids |
| L-leucine | 42.1 |
| L-isoleucine | 11.2 |
| L-valine | 13.0 |
| L-threonine | 10.8 |
| L-lysin hydrochloride | 12.6 |
| L-methionine | 3.1 |
| L-histidine hydrochloride 1 hydrate | 1.1 |
| L-phenylalanine | 5.6 |
| L-tryptophan | 0.5 |

### Experimental Example 1. Study of Influence of Composition of Example 1 on Muscular Endurance at the Time of Loading of Eccentric (Eccentric Muscle Contraction) Exercise

Male SD rats (purchased from CHARLES RIVER LABORATORIES JAPAN, INC. (Kanagawa)) were acclimation bred for 14 days, divided into 2 groups (n=4/group), and each group was fasted overnight. The composition of Example 1 was orally administered at 1 g/kg body weight to one of the group (group AA) and the same amount of purified water was orally administered to the other group (control group). After 30 min, electrical stimulation of 4.5 mA was given to the hindlimb tibialis anterior muscle of each rat by an ankle exercise device for small animal (manufactured by Bioresearch center Co. Ltd.), and an elongation stimulus was given ten times by changing the angle of ankle from 45° to 135° at a rate of 100 deg/sec. With the aforementioned eccentric exercise load as one set, a rest for 1 min was taken between each set of exercise loading and 10 sets of the exercise were performed. The muscle force of the hindlimb tibialis anterior muscle was measured every time one set of the exercise load was completed.

Two minutes before start of the above-mentioned exercise loading, the muscle force of hindlimb tibialis anterior muscle of each rat was measured and taken as the muscle force before exercise. Two minutes after completion of 10 sets of the exercise loading, muscle force was similarly measured and taken as the muscle force after exercise. Regarding the measurement results of muscle force, a t-test was performed between the group to which the composition of Example 1 was administered (group AA) and the control group.

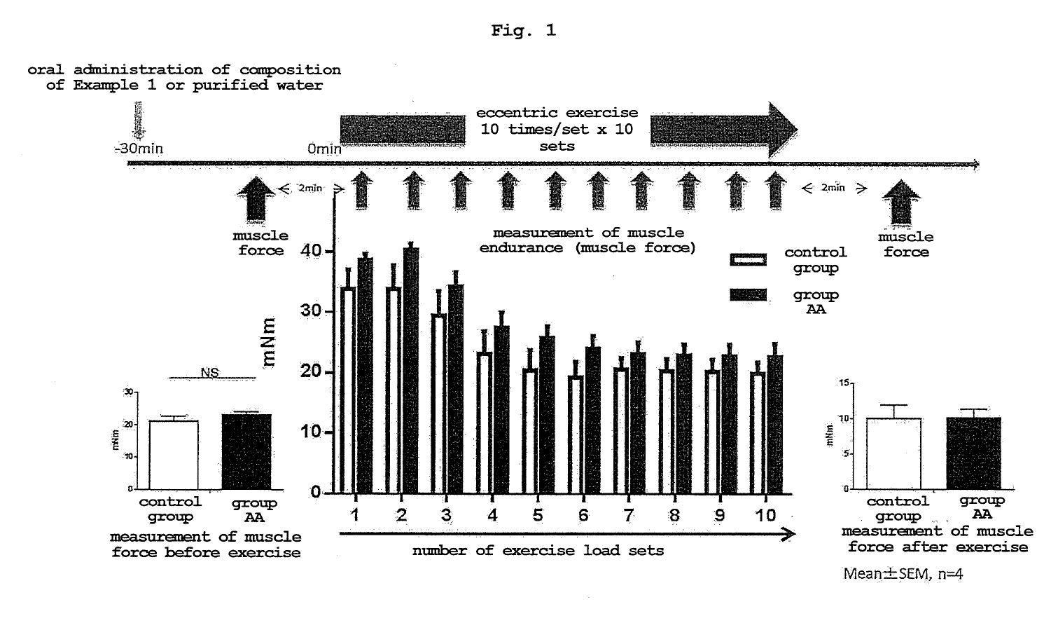

The measurement results of the muscle force before exercise and muscle force after exercise, and the muscle force after completion of each set of the above-mentioned exercise load are shown in FIG. 1 as mean±standard error of mean of 4 rats.

As shown in FIG. 1, a significant difference in the muscle force before exercise and muscle force after exercise was not found between the Example 1 composition administration group (group AA) and the control group. The decrease in muscle force by exercise loading was observed to be moderate in the group administered with the composition of Example 1 (group AA) than in the control group.

### Experimental Example 2. Study of Influence of Composition of Example 1 on Muscular Endurance at the Time of Loading of Concentric (Concentric Muscle Contraction) Exercise

Male SD rats (purchased from CHARLES RIVER LABORATORIES JAPAN, INC. (Kanagawa)) were acclimation bred for 14 days, divided into 2 groups (n=6/group), and each group was fasted overnight. The composition of Example 1 was orally administered at 1 g/kg body weight to one of the group (group AA) and the same amount of purified water was orally administered to the other group (control group). After 30 min, electrical stimulation of 4.5 mA was given to the hindlimb tibialis anterior muscle of each rat by an ankle exercise device for small animal (manufactured by Bioresearch center Co. Ltd.), and a concentric stimulus was given ten times by changing the angle of ankle from 135° to 45° at a rate of 100 deg/sec. With the aforementioned concentric exercise load as one set, a rest for 1 min was taken between each set of exercise loading and 10 sets of the exercise were performed. The muscle force of the hindlimb tibialis anterior muscle was measured every time one set of the exercise load was completed.

Two minutes before start of the above-mentioned exercise loading, the muscle force of hindlimb tibialis anterior muscle of each rat was measured and taken as the muscle force before exercise. Two minutes after completion of 10 sets of the exercise loading, muscle force was similarly measured and taken as the muscle force after exercise. Regarding the measurement results of muscle force, a t-test was performed between the group to which the composition of Example 1 was administered (group AA) and the control group.

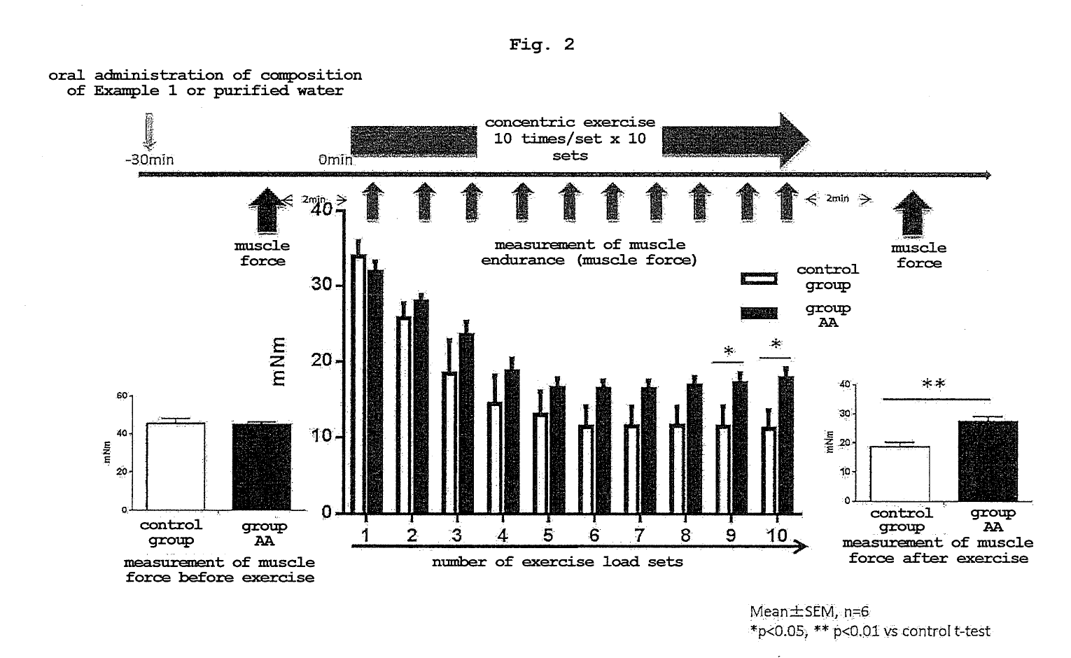

The measurement results of the muscle force before exercise and muscle force after exercise, and the muscle force after completion of each set of the above-mentioned exercise load are shown in FIG. 2 as mean±standard error of mean of 6 rats.

As shown in FIG. 2, a decrease in the muscle force after exercise was significantly (P<0.01) suppressed in the group to which the composition of Example 1 was administered (group AA), as compared to the control group. In the group to which the composition of Example 1 was administered (group AA), a decrease in the muscle force due to exercise loading was obviously mild as compared to the control group. As for the muscle force after completion of 9 and 10 sets of exercise loading, a decrease in the muscle force was significantly (P<0.05) suppressed in the group to which the composition of Example 1 was administered (group AA), as compared to the control group.

### Experimental Example 3. Study of Influence of Composition of Example 1 on Muscular Endurance at the Time of Loading of Eccentric (Eccentric Muscle Contraction) Exercise Under Different Exercise Load Conditions

Male SD rats (purchased from CHARLES RIVER LABORATORIES JAPAN, INC. (Kanagawa)) were acclimation bred for 14 days, divided into 2 groups (n=4/group), and each group was fasted overnight. The composition of Example 1 was orally administered at 1 g/kg body weight to one of the group (group AA) and the same amount of purified water was orally administered to the other group (control group). After 30 min, electrical stimulation of 4.5 mA was given to the hindlimb tibialis anterior muscle of each rat by an ankle exercise device for small animal (manufactured by Bioresearch center Co. Ltd.), and an elongation stimulus was given ten times by changing the angle of ankle from 45° to 90° at a rate of 100 deg/sec. With the aforementioned eccentric exercise load as one set, a rest for 1 min was taken between each set of exercise loading and 10 sets of the exercise were performed. The muscle force of the hindlimb tibialis anterior muscle was measured every time one set of the exercise load was completed.

Two minutes before start of the above-mentioned exercise loading, the muscle force of hindlimb tibialis anterior muscle of each rat was measured and taken as the muscle force before exercise. Two minutes after completion of 10 sets of the exercise loading, muscle force was similarly measured and taken as the muscle force after exercise.

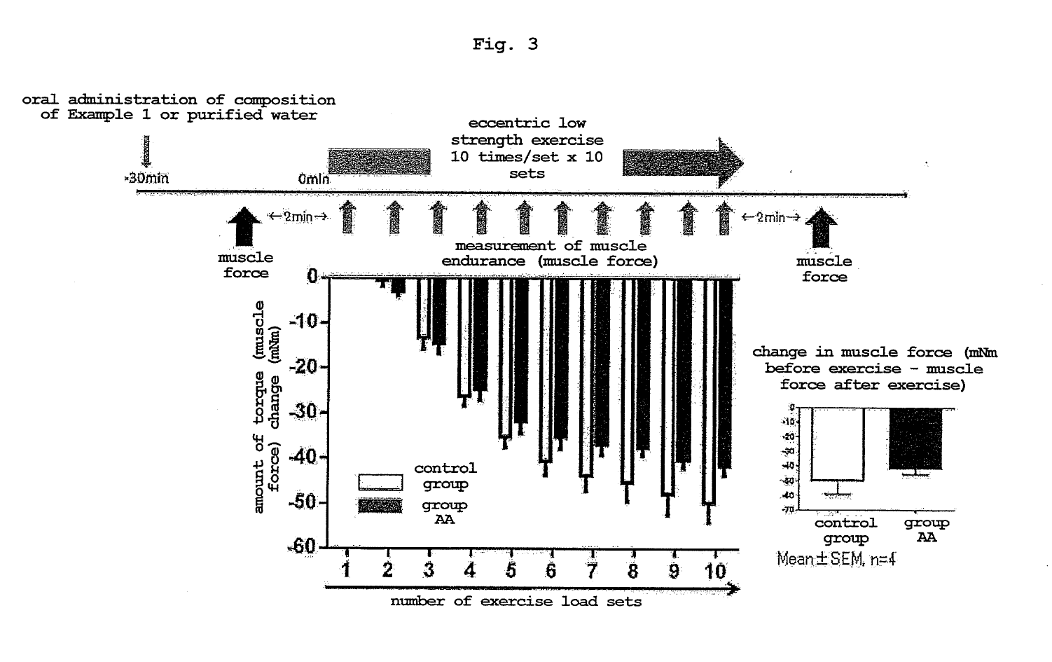

The amount of change in muscle force due to exercise (difference between muscle force before exercise and muscle force after exercise), and the amount of change in muscle force after the completion of each set of the above-mentioned exercise load (difference between muscle force before exercise and muscle force after completion of each set of the above-mentioned exercise load) were determined, and shown in FIG. 3 as mean±standard error of mean of 4 rats. Regarding the amount of change in muscle force due to exercise and the amount of change in muscle force after completion of each set of the above-mentioned exercise load, a t-test was performed between the group to which the composition of Example 1 was administered (group AA) and the control group.

As shown in FIG. 3, a significant difference in the amount of change in muscle force due to exercise and the amount of change in muscle force after completion of each set of the above-mentioned exercise load was not found between the Example 1 composition administration group (group AA) and the control group. The decrease in muscle force by exercise loading was observed to be moderate in the group administered with the composition of Example 1 (group AA) than in the control group, and the tendency was maintained until completion of the exercise loading. In addition, a tendency toward suppression of a decrease in muscle force after completion of exercise was found in the group to which the composition of Example 1 was administered (group AA) as compared to the control group.

From the results of Experimental Examples 1 to 3, administration once of the composition of Example 1 at 30 min before exercise showed suppression of a decrease in muscle force caused by resistance exercise load (particularly, concentric exercise load) and improvement of muscular endurance.

### Example 2. Composition for Improving Muscular Endurance

A given amount of each component was weighed to afford the composition shown in Table 2 and mixed to prepare the composition for improving muscular endurance of Example 2 (hereinafter to be referred to as “the composition of Example 2”).

TABLE 2

| | molar composition ratio (mol %) |
|---|---|
| | relative to total content of |
| component | essential amino acids |
| L-leucine | 41.6 |
| L-isoleucine | 14.2 |
| L-valine | 12.8 |
| L-threonine | 11.4 |
| L-lysin hydrochloride | 12.6 |
| L-phenylalanine | 7.4 |

### Experimental Example 4. Study of Influence of Composition of Example 2 on Muscular Endurance at the Time of Loading of Concentric (Concentric Muscle Contraction) Exercise

Male SD rats (purchased from CHARLES RIVER LABORATORIES JAPAN, INC. (Kanagawa)) were acclimation bred for 14 days, divided into 2 groups (n=4/group), and each group was fasted overnight. The composition of Example 2 was orally administered at 1 g/kg body weight to one of the group (group AB) and the same amount of purified water was orally administered to the other group (control group). After 30 min, electrical stimulation of 4.5 mA was given to the hindlimb tibialis anterior muscle of each rat by an ankle exercise device for small animal (manufactured by Bioresearch center Co. Ltd.), and a concentric stimulus was given ten times by changing the angle of ankle from 135° to 45° at a rate of 100 deg/sec. With the aforementioned concentric exercise load as one set, a rest for 1 min was taken between each set of exercise loading and 10 sets of the exercise were performed. The muscle force of the hindlimb tibialis anterior muscle was measured every time one set of the exercise load was completed.

Two minutes before start of the above-mentioned exercise loading, the muscle force of hindlimb tibialis anterior muscle of each rat was measured and taken as the muscle force before exercise. Two minutes after completion of 10 sets of the exercise loading, muscle force was similarly measured and taken as the muscle force after exercise.

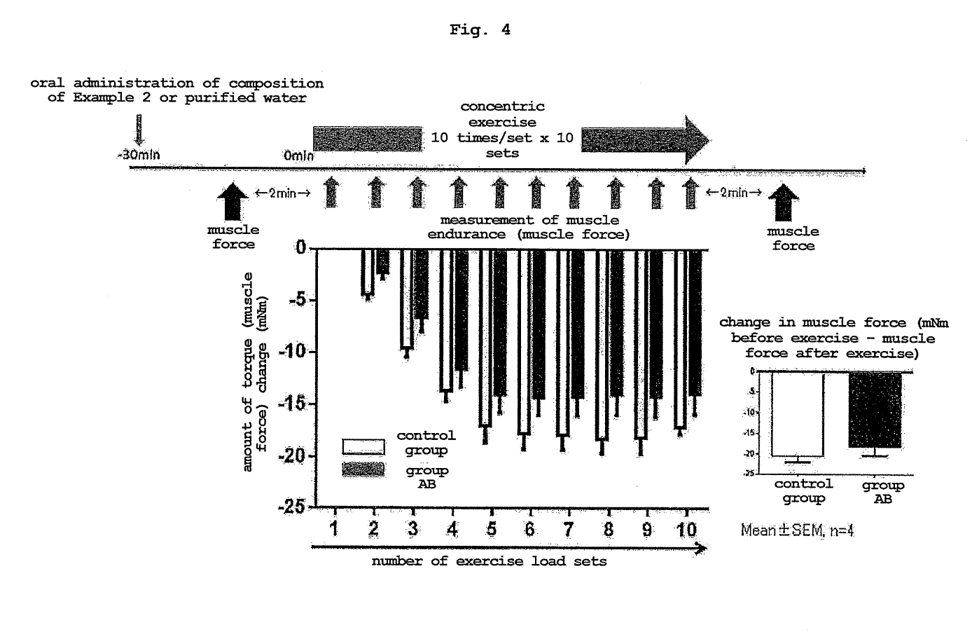

The amount of change in muscle force due to exercise (difference between muscle force before exercise and muscle force after exercise), and the amount of change in muscle force after the completion of each set of the above-mentioned exercise load (difference between muscle force before exercise and muscle force after completion of each set of the above-mentioned exercise load) were determined, and shown in FIG. 4 as mean±standard error of mean of 4 rats. Regarding the amount of change in muscle force due to exercise and the amount of change in muscle force after completion of each set of the above-mentioned exercise load, a t-test was performed between the group to which the composition of Example 2 was administered (group AB) and the control group.

As shown in FIG. 4, a significant difference in the amount of change in muscle force due to exercise and the amount of change in muscle force after completion of each set of the above-mentioned exercise load was not found between the Example 2 composition administration group (group AB) and the control group. The decrease in muscle force after completion of each set of the exercise load was moderate in the group administered with the composition of Example 2 (group AB) than in the control group, and the tendency was maintained until completion of the exercise loading. In addition, a tendency toward suppression of a decrease in muscle force after completion of exercise was found in the group to which the composition of Example 2 was administered (group AB) as compared to the control group.

From the results of Experimental Example 4, administration once of the composition of Example 2 at 30 min before exercise showed suppression of a decrease in muscle force caused by concentric exercise load and improvement of muscular endurance.

### INDUSTRIAL APPLICABILITY

As described in detail above, according to the present invention, a composition for improving muscular endurance that can efficiently improve muscular endurance conveniently in a short period of time can be provided.

That is, the composition for improving muscular endurance of the present invention can improve muscle endurance by suppressing a decrease in muscle force caused by exercise load.

Furthermore, the composition for improving muscular endurance of the present invention favorably suppresses a decrease in muscle force and favorably improves muscular endurance against exercise load by injection or administration once before exercise.

The composition for improving muscular endurance of the present invention is particularly effective for suppressing a decrease in muscle force and improving muscular endurance when a resistance exercise is performed in which the load is concentrated on the target muscle, and further effective for suppressing a decrease in muscle force and improving muscular endurance when concentric exercise is performed.

Therefore, the composition for improving muscular endurance of the present invention is preferably utilized by not only athletes requiring improvement of muscular endurance but also patients requiring exercise therapy and continuous rehabilitation, elderly people and middle-aged people showing a decrease in muscle force and muscular endurance and the like.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition for improving muscular endurance, comprising:
one or more kinds of essential amino acids other than leucine; and
not less than 35 mol % of leucine, relative to the total content of essential amino acids.

2. The composition according to claim 1, wherein the content of leucine is 35 mol % to 66 mol %, relative to the total content of essential amino acids.

3. The composition according to claim 1, wherein said is one or more kinds of essential amino acids other than leucine are isoleucine and valine.

4. The composition according to claim 3, wherein a molar composition ratio of the content of each amino acid relative to the total content of essential amino acids falls within the following numerical values:
leucine, 35 mol % to 66 mol %
isoleucine, 5 mol % to 15 mol %
valine, 5 mol % to 15 mol %.

5. The composition according to claim 1, wherein said one or more kinds of essential amino acids other than leucine are isoleucine, valine, threonine, lysine, and phenylalanine.

6. The composition according to claim 5, wherein a molar composition ratio of the content of each amino acid relative to the total content of essential amino acids falls within the following numerical values:
leucine, 35 mol % to 66 mol %
isoleucine, 5 mol % to 15 mol %
valine, 5 mol % to 15 mol %
threonine, 7 mol % to 14 mol %
lysine, 8 mol % to 16 mol %
phenylalanine, 2.5 mol % to 8 mol %.

7. The composition according to claim 1, wherein said one or more kinds of essential amino acids other than leucine are isoleucine, valine, threonine, lysine, methionine, histidine, phenylalanine, and tryptophan.

8. The composition according to claim 7, wherein a molar composition ratio of the content of each amino acid relative to the total content of essential amino acids falls within the following numerical values:
0.15 leucine, 35 mol % to 66 mol %
isoleucine, 5 mol % to 15 mol %
valine, 5 mol % to 15 mol %
threonine, 7 mol % to 14 mol %
lysine, 8 mol % to 16 mol %
methionine, 2 mol % to 10 mol %
histidine, 0.1 mol % to 3.5 mol %
phenylalanine, 2.5 mol % to 8 mol %
tryptophan, 0.1 mol % to 2 mol %.

9. The composition according to claim 1, which is a pharmaceutical product.

10. The composition according to claim 1, which is a food.

11. A method for improving muscular endurance, comprising ingestion by or administration to a subject animal in need of improvement in muscular endurance of an effective amount of a composition comprising
one or more kinds of essential amino acids other than leucine; and
not less than 35 mol % of leucine, relative to the total content of essential amino acids.

12. The method according to claim 11, wherein said composition comprises leucine in an amount of 35 mol % to 66 mol %, relative to the total content of essential amino acids.

13. The method according to claim 11, wherein said one or more kinds of essential amino acids other than leucine are isoleucine and valine.

14. The method according to claim 13, wherein a molar composition ratio of the content of each amino acid relative to the total content of essential amino acids in said composition falls within the following numerical values:
leucine, 35 mol % to 66 mol %
isoleucine, 5 mol % to 15 mol %
valine, 5 mol % to 15 mol %.

15. The method according to claim 11, wherein said one or more kinds of essential amino acids other than leucine are isoleucine, valine, threonine, lysine, and phenylalanine.

16. The method according to claim 15, wherein a molar composition ratio of the content of each amino acid relative to the total content of essential amino acids in said composition falls within the following numerical values:
leucine, 35 mol % to 66 mol %
isoleucine, 5 mol % to 15 mol %
valine, 5 mol % to 15 mol %
threonine, 7 mol % to 14 mol %
lysine, 8 mol % to 16 mol %
phenylalanine, 2.5 mol % to 8 mol %.

17. The method according to claim 11, wherein said one or more kinds of essential amino acids other than leucine are isoleucine, valine, threonine, lysine, methionine, histidine, phenylalanine, and tryptophan.

18. The method according to claim 17, wherein a molar composition ratio of the content of each amino acid relative to the total content of essential amino acids in said composition falls within the following numerical values:
leucine, 35 mol % to 66 mol %
isoleucine, 5 mol % to 15 mol %
valine, 5 mol % to 15 mol %
threonine, 7 mol % to 14 mol %
lysine, 8 mol % to 16 mol %
methionine, 2 mol % to 10 mol %
histidine, 0.1 mol % to 3.5 mol %
phenylalanine, 2.5 mol % to 8 mol %
tryptophan, 0.1 mol % to 2 mol %.

19. The method according to claim 11, which improves muscular endurance against resistance exercise load.

20. The composition according to claim 19, wherein said resistance exercise is concentric exercise.

21. The method according to claim 11, wherein said composition is ingested or administered once before exercise.
